(19) United States
(12) Patent Application Publication
(10) Pub. No.: US 2006/0110417 A1
(43) Pub. Date: May 25, 2006

(54) Beauty products and methods

(76) Inventor: Lori Hamlin, (US)

Correspondence Address: FISH & RICHARDSON PC, P.O. BOX 1022, MINNEAPOLIS, MN, 55440-1022, US

(21) Appl. No.: 10/997,148

(22) Filed: Nov. 24, 2004

(51) Int. Cl. G06Q 10/00; A61K 8/02

(52) U.S. Cl. 424/401; 705/2

(57) ABSTRACT

A beauty kit including a beauty product having a beauty product identifier, and at least one template having a facial feature including one or more facial feature identifiers arranged in a predetermined pattern. The facial feature identifiers correspond to the beauty product identifier.

### TECHNICAL FIELD

This invention relates to beauty products and methods.

### BACKGROUND

Beauty products, such as cosmetics and skin care supplies, can be used to enhance the appearance of men and women alike. Beauty products, for example, can be used for daily applications, as well as for special events, such as costume parties, television appearances, and photo shoots. The selection of beauty products and/or the techniques used to apply beauty products can depend on the event for which the wearer of the beauty product is preparing.

### SUMMARY

In one aspect of the invention, a beauty kit includes beauty products having a beauty product identifier, applicators having an applicator identifier, and templates having a facial feature corresponding to a facial feature of a user. The facial feature of the templates includes facial feature identifiers arranged in a pattern. The facial feature identifiers match at least one of the beauty product identifiers and at least one of the applicator identifiers.

In another aspect, a beauty kit includes a template having a facial feature thereon. The facial feature of the template corresponds to a facial feature of a user, and includes one or more facial feature identifiers arranged in a pattern. The beauty kit also includes a beauty product having a beauty product identifier matching the one or more facial feature identifiers.

In an additional aspect, a beauty template includes a first facial feature having one or more facial feature identifiers arranged in a pattern corresponding to a pattern in which a beauty product is to be applied to a corresponding facial feature of a user.

In another aspect, a beauty product for use with a beauty template having one or more facial feature identifiers arranged in a predetermined configuration about a facial feature of the template includes a beauty product identifier matching the one or more facial feature identifiers of the beauty template.

In yet another aspect, a method of using a beauty kit includes selecting a template including a facial feature thereon that corresponds to a facial feature of a user, the facial feature of the template including at least one facial feature identifier; selecting a beauty product comprising a beauty product identifier matching the at least one facial feature identifier; and applying the beauty product to a region of the facial feature of the user corresponding to a region of the facial feature of the template on which the at least one facial feature identifier is arranged.

In an additional aspect, a method includes selecting a beauty product including a beauty product identifier corresponding to an identifier of a facial feature of a beauty template; and applying the beauty product to a facial feature of the user corresponding to the facial feature of the beauty template.

In another aspect, a method of making a beauty template includes providing a facial feature on a sheet of material. The facial feature includes at least at least one facial feature identifier matching an identifier of at least one beauty product.

Embodiments may include one or more of the following features.

In some embodiments, the pattern of the facial feature identifiers corresponds to a pattern in which the beauty product is to be applied to the facial feature of the user. In certain embodiments, a region of the facial feature of the user to which the beauty product is to be applied is substantially covered by the facial feature identifiers.

In some embodiments, the facial feature of the template includes multiple different facial feature identifiers arranged in multiple different patterns. In some embodiments, each of the beauty product identifiers corresponds to a different beauty product.

In certain embodiments, the beauty kit further includes instructions (e.g. audio and/or video instructions) describing a manner of applying the beauty products to the user.

In some embodiments, the facial feature of the template has a shape substantially similar to the corresponding facial feature of the user.

In certain embodiments, the applicators include a brush, a sponge, a cotton ball, and/or a cotton swab. In some embodiments, the facial feature includes an eye, a lip, and/or a cheek. In certain embodiments, the beauty product includes powder, foundation, blush, concealer, eyeshadow, lipstick, lipgloss, eyeliner, lipliner, mascara, and/or bronzer.

In some embodiments, the beauty kit further includes an applicator including an applicator identifier, and the one or more facial feature identifiers match the applicator identifier.

In certain embodiments, the facial feature of the template includes multiple different facial feature identifiers arranged in multiple different patterns. In some embodiments, the beauty kit includes multiple beauty products, each beauty product including a different beauty product identifier, and each of the different beauty product identifiers match at least one of the facial feature identifiers.

In certain embodiments, the facial feature identifiers include a letter, a number, a character, and/or a symbol.

In some embodiments, the beauty template is preselected to provide the user with a target look. In certain embodiments, the beauty product includes a preselected color.

In some embodiments, the beauty template includes a second facial feature including at least one second facial feature identifier thereon, the at least one second facial feature identifier corresponding to another facial feature of the user.

In certain embodiments, the beauty template comprises an adherable surface. In some embodiments, the template is adhered to a surface (e.g., the surface of a mirror).

In certain embodiments, an applicator including an applicator identifier matching the at least one facial feature identifier is selected, and the beauty product is applied to the facial feature of the user using the selected applicator.

In certain embodiments, the template includes a plurality of facial features, each of the facial features of the template corresponding to a facial feature of the user, and each of the facial features of the template including at least one facial feature identifier. In some embodiments, the method further includes selecting at least one beauty product including a beauty product identifier matching the at least one facial feature identifier, and selecting at least one applicator including an applicator identifier matching the at least one facial feature identifier, and applying the at least one beauty product to the facial features of the user that correspond to the facial features of the template.

In certain embodiments, a template including a facial feature having a shape substantially similar to a corresponding facial feature of the user is selected.

In some embodiments, the beauty product is applied to the facial feature of the user in a pattern corresponding to the pattern of the facial feature identifiers. In certain embodiments, selecting the beauty product includes matching the beauty product identifier with the facial feature identifier. In some embodiments, an applicator including an applicator identifier matching the beauty product and facial feature identifiers is also selected.

The embodiments may include one or more of the following advantages.

In some embodiments, the user can quickly learn appropriate techniques for applying beauty products. In certain embodiments, the methods and products can be used to teach the user appropriate techniques for applying beauty products without the assistance an outside consultant or trainer. In some embodiments, the methods and products allow the user to learn techniques for applying beauty products within a private setting.

In certain embodiments, the methods and products allow the user to use appropriate color schemes and/or appropriate types of beauty products. In some embodiments, the methods and products allow the user to apply beauty products to appropriate areas of his/her body (e.g., appropriate areas of his/her face).

In certain embodiments, the methods and/or products can be utilized for men and/or women of any age, race, skin tone, and/or skin type.

In some embodiments, the beauty products are preselected to compliment the user's skin tone, skin color, eye color, hair color, and/or age. In some embodiments, the beauty products are preselected to provide a targeted look (e.g., a daytime look, an evening look, a fall look, a winter look, a spring look, a dramatic look, a mature look, a formal look, and/or a costume look).

Other features and advantages of the invention will be apparent from the description, the drawings, and the claims.

### DESCRIPTION OF DRAWINGS

FIG. 1 is a schematic view of a beauty kit.

FIG. 2 is an enlarged view of region A of FIG. 1.

FIG. 3 is an enlarged view of region B of FIG. 1.

Like reference symbols in the various drawings indicate like elements.

### DETAILED DESCRIPTION

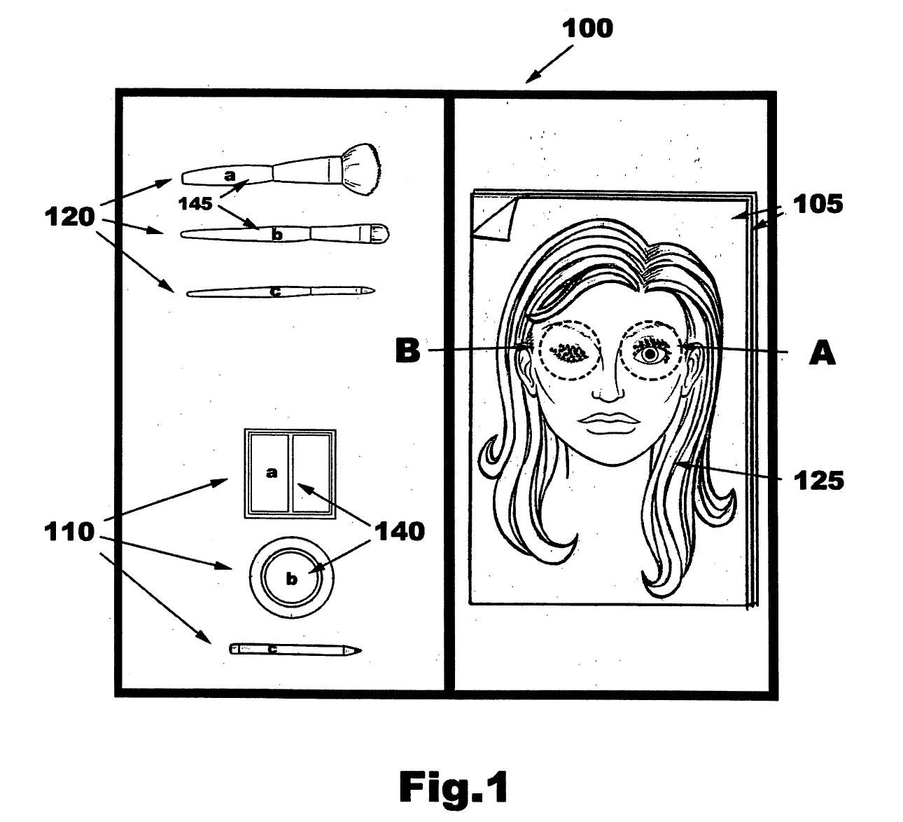

Referring to FIG. 1, a beauty kit 100 includes multiple templates 105, beauty products 110, and beauty product applicators 120. The templates 105, the beauty products 110, and the applicators 120 include identifiers 135, 140, 145, respectively. One of the templates 105 can be selected by a user to help the user of the beauty kit 100 apply one or more of the beauty products 110 to designated regions of his/her face. The selected template 105 can also be used to help the user select appropriate beauty product(s) 110 and, in some cases, appropriate applicator(s) 120 with which to apply the beauty product(s) 110. For example, the user can select the beauty product 110 and the applicator 120 having identifiers 140, 145, respectively, that match the identifier 135 on a facial feature of the template 105. Using the selected applicator 120, the user can then apply the selected beauty product 110 to those regions of his/her facial feature that correspond to the regions of the template on which the identifiers 135 are located.

The beauty products 110 can be cosmetics and/or skin care products, for example. Examples of cosmetics include powder, foundation, blush, concealer, eyeshadow, lipstick, lipgloss, eyeliner, lipliner, mascara, and bronzer. Examples of skin care products include moisturizer, cleanser, toner, exfoliator, mask, eye cream, throat cream, serums, hand creams, and sunscreen.

The beauty products 110, as noted above, include beauty product identifiers 140. Each of the beauty products 110 can be labeled with the beauty product identifiers 140 in any of various ways. For example, a housing of the beauty product 110 can be engraved, painted, stamped, and/or printed to include the beauty product identifier 140. A sticker including the beauty product identifier 140 can similarly be adhered to the housing of the beauty product 110. Alternatively or additionally, a film or sheet including the beauty product identifier can be situated within the housing of the beauty product (e.g., disposed on top of the beauty product within the housing). In some embodiments, the beauty product 110 itself is labeled. For example, the beauty product 110 can be “hot pressed” to include a depression in the shape of the beauty product identifier 140.

The applicators 120 can be used to apply the beauty products 110 to the face of the user. Examples of applicators include cosmetic brushes, sponges, cotton pads, cotton balls, and cotton swabs. The applicators 120, as noted above, include applicator identifiers 145. Each of the applicators 120 can be labeled with the applicator identifiers 145 using any of the various techniques. For example, the applicator 120 can be engraved, painted, stamped, and/or printed to include the applicator identifier 145. A sticker including the applicator identifier 145 can similarly be adhered to the applicator 120.

Each of the templates 105 includes a facial image 125 that includes a printed outline of eyes, cheeks, lips, and/or a nose. The shape of the eyes differs among the multiple templates 105. For example, each of the templates 105 can include eyes shaped differently than the eyes of the other templates. Examples of eye shapes include round, almond, deep set, vanishing lid, close set, and wide set. The user can select the template 105 having eyes that most closely resemble the eyes of the user (e.g., eyes having a shape most similar shape to the shape of the user's eyes). By selecting the template 105 having an eye shape that most closely resembles the eye shape of the user, for example, the user can learn techniques for applying one or more of the beauty products 110 to eyes having his/her shape.

The eyes of the facial image 125 of the template 105 include regions that correspond to regions of the user's eye. One or more of these regions of the template can include a patterned array of identifiers 135. The identifiers 135, for example, can be arranged in a pattern corresponding to a pattern in which the user can apply one or more of the beauty products 110 to corresponding regions of his/her face. The identifiers can include one or more letters, numbers, characters, and/or symbols.

The beauty kit 100 can be used to apply one or more of the beauty products 110 to the user's face in a desired manner. The user first selects the template 105 having eyes that best correspond to the user's eye shape (e.g., the template that has eyes of a shape most similar to the user's eye shape). For example, a user having almond shaped eyes may select the template 105 with almond shaped eyes. As noted above, by selecting the template 105 having eyes of a shape most similar to the user's eye shape, the user can learn techniques that are particularly appropriate for applying beauty products to eyes of his/her shape.

After selecting the template 105, the user locates one of the patterned array of identifiers 135 included on the eye region of the template 105. The identifiers 135, as described above, are included on those regions of the template 105 corresponding to the eye regions of the user to which the beauty product(s) 110 is/are to be applied.

The user then selects the beauty product(s) 110 that include beauty product identifiers 140 matching the located identifiers 135. The user also selects the applicator(s) 120 having applicator identifiers 145 that match the located identifiers 135.

After selecting the beauty product(s) 110 and the applicator(s) 120, the user applies the selected beauty product(s) 110 to the regions of his/her face that correspond to the regions of the facial image 125 including the located identifiers 135. As noted above, the identifiers 135 can be arranged about the eye regions in different patterns depending on the targeted look. The user can apply the beauty product according to the pattern of the reference symbols. For example, the user can apply the beauty product(s) 110 to those regions of his/her eyes corresponding to regions of the template 105 covered by the identifiers 135.

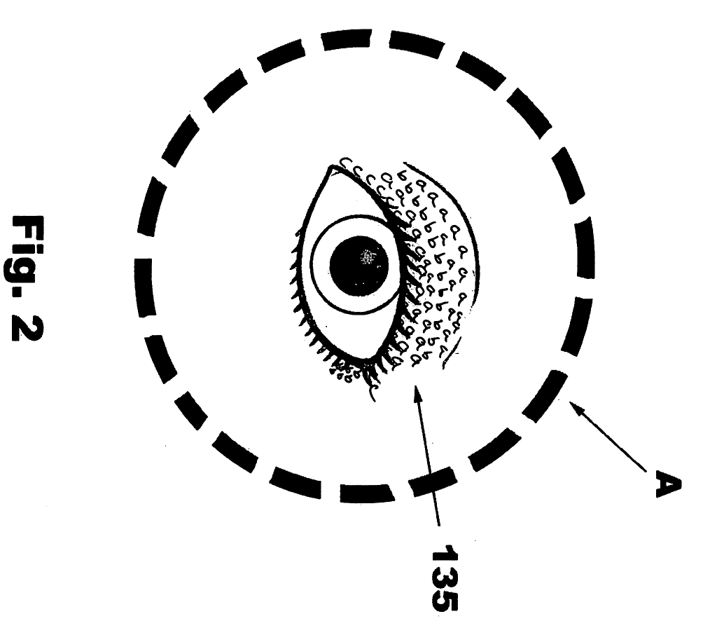

FIG. 2 illustrates an exemplary pattern in which the identifiers 135 can be arranged about the eye region of the template 105. In the embodiment shown, a first set of the identifiers 135 (represented as “a”) are arranged to substantially cover the eyelid. The beauty product 110 (e.g., eye shadow) with the beauty product identifier 140 matching the first set of the identifiers 135 (i.e., the beauty product 110 labeled with an “a”) is applied to those regions of the eyelid with the applicator 120 having the applicator identifier 145 matching the first set of the identifiers 135 (i.e., the applicator 120 labeled with an “a”). A second set of the identifiers 135 (represented as “b”) is included on a crease defined between the eyelid and the brow bone and on an outer corner region beneath the eye. The beauty product 110 (e.g., a second type of eye shadow) having the beauty product identifier 140 matching the second set of the identifiers 135 (i.e., the beauty product 110 labeled with a “b”) can be applied to these regions using the applicator 120 having the applicator identifier 145 matching the second set of the identifiers 135 (i.e., the applicator 120 labeled with a “b”). A third set of identifiers 135 (represented as “c) is included on a region positioned slightly above the eyelashes and extending along the eyelid. The beauty product 110 (e.g., eyeliner) having the beauty product identifier 140 matching the second set of the identifiers 135 (i.e., the beauty product 110 labeled with a “c”) can be applied to this region using the applicator 120 having the applicator identifier 145 matching the second set of the identifiers 135 (i.e., the applicator 120 labeled with a “c”).

Other embodiments are also possible.

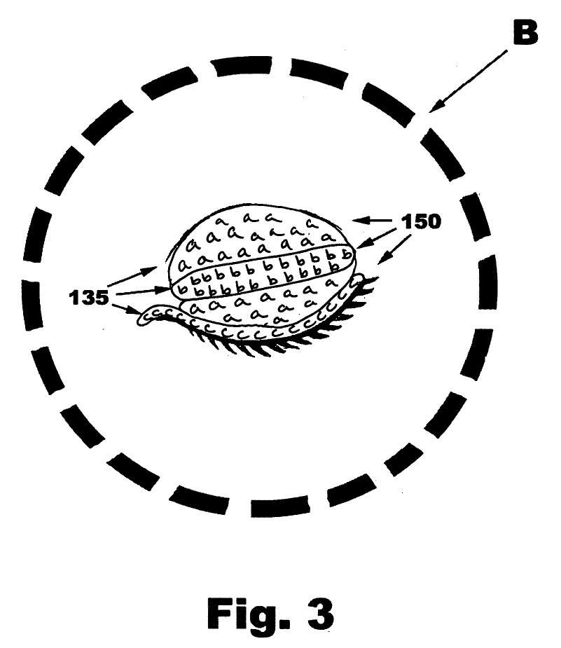

While the facial feature identifiers 135 of the embodiments above are arranged on the template 105 in patterned arrays, the identifiers 135 can alternatively or additionally be included in other arrangements. Referring to FIG. 3, for example, regions 150 corresponding to regions of the user to which the beauty product(s) 110 is/are to be applied are demarcated with an outline. Each of the demarcated regions 150 includes one or more of the identifiers 135. Thus, the user can apply the beauty product(s) 110 having beauty product identifiers 140 that match the identifiers 135 to regions of his/her face corresponding to the demarcated regions 150 of the template 105. In addition to or as an alternative to outlining, the regions 150 can be demarcated using other techniques, such as coloring and/or shading.

In some embodiments, other facial features (e.g., lips, cheeks, and/or nose) of the template 105 can alternatively or additionally include regions with identifiers. Thus, one or more of the beauty products 110 can be applied to the other facial features using techniques similar to those discussed above. In some embodiments, multiple facial features of the template 105 include identifiers. Accordingly, the user can apply one or more of the beauty products 110 to multiple different regions of his/her face.

While the embodiments discussed above included selecting the template 105 based on the eye shape of the user, the template 105 can alternatively or additionally be selected based on other characteristics of the user. For example, the template 105 can be selected based on the shape of other facial features of the user, such as the user's cheeks, lips, and/or a nose. For example, the user can select the template having cheeks, lips, and/or a nose of similar shape to the cheeks, lips, and/or nose of the user. Lips, for example, can be bow-shaped, full, small, thin, uneven, natural, bee-stung, and/or unilip. In certain embodiments, the user can select the template having a face shape that best corresponds to the shape of the user's face. For example the user can select the template having a facial image of a shape most similar to the shape of the user's face. Examples of face shapes include long, square, oval, round, and heart-shaped.

In some embodiments, the beauty kit 100 includes a set of instructions that provide the user with beauty product application tips. For example, the instructions can explain in a step-by-step process how to apply the beauty product(s) 110 to particular regions of the user's face. The instructions, for example, can explain appropriate brush strokes that can be used to apply certain beauty products (e.g., appropriate motions and pressure that can be used). The instructions can similarly explain how much of the beauty product(s) 110 to apply to the face of the user. Further, the instructions can provide other application tips to achieve particular types of looks (e.g., how to make the eyes look more open, how to make lips look fuller). In some embodiments, the beauty kit can include audio and/or video instructions.

In certain embodiments, the beauty products 110 can be preselected to include colors that compliment one another. As a result, the beauty kit 100 can help to prevent the user from applying colors that do not look well with one another. The beauty products can alternatively or additionally be preselected to supply the user with an appropriate combination of beauty product types (e.g., powders, lotions, gels, etc.) As a result, the beauty kit 100 can help to prevent the user from mixing inappropriate types of beauty products.

In some embodiments, the beauty products 110 can be preselected to provide the user with a targeted look. For example, the beauty products 110 can be selected to provide a daytime look, an evening look, a fall look, a winter look, a spring look, a summer look, a dramatic look, a mature look, and/or a costume look. In order to create these looks, for example, the beauty kit 100 can include beauty products of preselected colors and the template 105 can include a preselected arrangement of the identifiers 135 about various regions of the facial image on the template. The colors, for example, can be preselected based on the user's skin tone, the season, the trend.

In certain embodiments, the beauty products 110 can be preselected to compliment one or more characteristics of the user, such as hair color, eye color, and/or skin color.

In some embodiments, the beauty products 110 and/or the applicators 120 can be replaced or supplemented with other beauty products and/or applicators. For example, the user may acquire additional beauty products and/or applicators that can be used in place of the original beauty products and/or applicators. This can be beneficial, for example, when the user desires a color change (e.g., change of seasons) and/or a style change (e.g., formal rather than informal). In some embodiments, the beauty kit can be supplemented with additional products, applicators, and/or templates. For example, as the user becomes more skilled in beauty product application, he/she may supplement the beauty kit to produce additional looks.

In some embodiments, the beauty kit 100 includes multiple templates that include one facial feature. The user can select a template that is most appropriate for multiple facial features. For example, the user can select the template that includes an eye shape most similar to his/her own eye shape, the template that includes a lip shape most similar to his/her own lip shape, the template that includes a cheek shape most similar to his/her own cheek shape, and/or the template that includes a face shape most similar to his/her own face shape.

In certain embodiments, the applicator and the beauty product are combined. Examples of such combinations include eyeliner pencils and lip liner pencils. In such embodiments, the combined beauty product/applicator can include an identifier. In some embodiments, application of the beauty product does not require the use of an applicator. In such embodiments, the user can apply the appropriate beauty products with his/her finger, for example. Thus, in some embodiments, the beauty kit does not include any applicators.

In some embodiments, one or more of the applicators 120 can be appropriate for the application of more than one of the beauty products 110. For example, one of the applicators may be an appropriate tool with which to apply eye shadow to the eye region of the user and blush to the cheek region of the user. Thus, some of the applicators 120 may include multiple different applicator identifiers 140.

In some embodiments, the templates 105 are made of one or more materials that can be releasably adhered to a surface, such as a mirror. By applying the template to a mirror, for example, the user can easily reference the template while applying beauty products to his/her face. The template 105, for example, can be made of any of various plastic materials that are capable of adhering to a surface. Alternatively or additionally, the template 105 can include a layer of adhesive on its backside to allow the template to be adhered to the surface.

While the embodiments discussed above involve applying beauty products to the user's face, the beauty kit can alternatively or additionally be used for applying beauty products to any of various other regions of the body. In such embodiments, the template of the beauty kit can include a printed image of a body part other than a face. Particular regions of the body part can include identifiers, as described above, to help the user apply the beauty products to corresponding regions of his/her body.

Other embodiments are within the claims.

1. A beauty kit, comprising:
a plurality of beauty products, at least some of the beauty products comprising a beauty product identifier;
a plurality of applicators, at least some of the applicators comprising an applicator identifier; and
a plurality of templates, at least some of the templates comprising a facial feature corresponding to a facial feature of a user, the facial feature of the templates comprising a plurality of facial feature identifiers arranged in a pattern, the facial feature identifiers matching at least one of the beauty product identifiers and at least one of the applicator identifiers.

2. The beauty kit of claim 1, wherein the pattern of the facial feature identifiers corresponds to a pattern in which the beauty product is to be applied to the facial feature of the user.

3. The beauty kit of claim 2, wherein a region of the facial feature of the user to which the beauty product is to be applied is substantially covered by the facial feature identifiers.

4. The beauty kit of claim 1, wherein the facial feature of the template comprises multiple different facial feature identifiers arranged in multiple different patterns.

5. The beauty kit of claim 1, further comprising instructions describing a manner of applying the beauty products to the user.

6. The beauty kit of claim 1, wherein the facial feature of the template has a shape substantially similar to the corresponding facial feature of the user.

7. The beauty kit of claim 1, wherein the applicators comprise one or more members selected from the group consisting of a brush, a sponge, a cotton ball, and a cotton swab.

8. The beauty kit of claim 1, wherein the facial feature comprises a member selected from the group consisting of an eye, a lip, and a cheek.

9. The beauty kit of claim 1, wherein the beauty product comprises one or more members selected from the group consisting of powder, foundation, blush, concealer, eyeshadow, lipstick, lipgloss, eyeliner, lipliner, mascara, and bronzer.

10. A beauty kit, comprising:
a template comprising a facial feature thereon, the facial feature of the template corresponding to a facial feature of a user, the facial feature of the template comprising one or more facial feature identifiers arranged in a pattern; and
a beauty product comprising a beauty product identifier matching the one or more facial feature identifiers.

11. The beauty kit of claim 10, wherein the facial feature of the template has a shape substantially similar to a corresponding facial feature of a user.

12. The beauty kit of claim 10, further comprising an applicator comprising an applicator identifier, the one or more facial feature identifiers matching the applicator identifier.

13. The beauty kit of claim 10, wherein the beauty product applicator comprises one or more members selected from the group consisting of a brush, a sponge, a cotton ball, and a cotton swab.

14. The beauty kit of claim 10, further comprising instructions explaining a manner in which to apply the beauty product to the corresponding facial feature of the user.

15. The beauty kit of claim 14, wherein the instructions comprise audio instructions.

16. The beauty kit of claim 10, wherein the pattern in which the one or more facial feature identifiers are arranged corresponds to a pattern in which the beauty product is to be applied to the corresponding facial feature of the user.

17. The beauty kit of claim 10, wherein the facial feature of the template comprises multiple different facial feature identifiers, each of the beauty product identifiers corresponding to a different beauty product.

18. The beauty kit of claim 10, wherein the facial feature of the template comprises multiple different facial feature identifiers arranged in multiple different patterns.

19. The beauty kit of claim 18, wherein the beauty kit comprises multiple beauty products, each beauty product comprising a different beauty product identifier, each of the different beauty product identifiers matching at least one of the facial feature identifiers.

20. The beauty kit of claim 10, wherein the facial feature comprises a member selected from the group consisting of an eye, a lip, and a cheek.

21. The beauty kit of claim 10, wherein the beauty product comprises one or more members selected from the group consisting of powder, foundation, blush, concealer, eyeshadow, lipstick, lipgloss, eyeliner, lipliner, mascara, and bronzer.

22. The beauty kit of claim 10, wherein the facial feature identifiers comprise a member selected from the group consisting of a letter, a number, a character, and a symbol.

23. A beauty template comprising a first facial feature comprising one or more facial feature identifiers arranged in a pattern corresponding to a pattern in which a beauty product is to be applied to a corresponding facial feature of a user.

24. The beauty template of claim 23, wherein the beauty template is preselected to provide the user with a target look.

25. The beauty template of claim 24, wherein the beauty product comprises a beauty product identifier matching the one or more facial feature identifiers of the template.

26. The beauty template of claim 24, wherein the beauty product comprises a preselected color.

27. The beauty template of claim 23, wherein the first facial feature has a shape substantially similar to a shape of the corresponding facial feature of the user.

28. The beauty template of claim 23, wherein the beauty template comprises a second facial feature comprising at least one second facial feature identifier thereon, the at least one second facial feature identifier corresponding to another facial feature of the user.

29. The beauty template of claim 23, wherein the beauty template comprises an adherable surface.

30. A beauty product for use with a beauty template having one or more facial feature identifiers arranged in a predetermined configuration about a facial feature of the template, the beauty product comprising a beauty product identifier matching the one or more facial feature identifiers of the beauty template.

31. The beauty product of claim 30, wherein the beauty product comprises one or more members selected from the group consisting of powder, foundation, blush, concealer, eyeshadow, lipstick, lipgloss, eyeliner, lipliner, mascara, and bronzer.

32. The beauty product of claim 30, wherein the beauty product comprises a preselected color to provide a preselected look.

33. A method of using a beauty kit, the method comprising:
selecting a template comprising a facial feature thereon that corresponds to a facial feature of a user, the facial feature of the template comprising at least one facial feature identifier;
selecting a beauty product comprising a beauty product identifier matching the at least one facial feature identifier; and
applying the beauty product to a region of the facial feature of the user corresponding to a region of the facial feature of the template on which the at least one facial feature identifier is arranged.

34. The method of claim 33, further comprising selecting an applicator comprising an applicator identifier matching the at least one facial feature identifier, and applying the beauty product to the facial feature of the user using the selected applicator.

35. The method of claim 33, further comprising adhering the template to a surface.

36. The method of claim 33, wherein the facial feature of the template comprises a plurality of facial feature identifiers.

37. The method of claim 36, wherein the plurality of facial feature identifiers includes at least two different facial feature identifiers.

38. The method of claim 37, wherein each of the different facial feature identifiers corresponds to a different beauty product.

39. The method of claim 36, wherein the facial feature identifiers are arranged to substantially cover the region of the facial feature of the template corresponding to the region of the facial feature of the user to which the beauty product is to be applied.

40. The method of claim 33, wherein the template comprises a plurality of facial features, each of the facial features of the template corresponding to a facial feature of the user, and each of the facial features of the template comprising at least one facial feature identifier.

41. The method of claim 40, further comprising selecting at least one beauty product comprising a beauty product identifier matching the at least one facial feature identifier, and selecting at least one applicator comprising an applicator identifier matching the at least one facial feature identifier, and applying the at least one beauty product to the facial features of the user that correspond to the facial features of the template.

42. The method of claim 33, wherein selecting the template comprises selecting a template comprising a facial feature having a shape substantially similar to a corresponding facial feature of the user.

43. The method of claim 33, wherein the facial feature comprises a member selected from the group consisting of an eye, a lip, and a cheek.

44. The method of claim 33, wherein the beauty product comprises one or more members selected from the group consisting of powder, foundation, blush, concealer, eyeshadow, lipstick, lipgloss, eyeliner, lipliner, mascara, and bronzer.

45. A method comprising:
selecting a beauty product comprising a beauty product identifier corresponding to an identifier of a facial feature of a beauty template; and
applying the beauty product to a facial feature of the user corresponding to the facial feature of the beauty template.

46. The method of claim 45, wherein the beauty template comprises a plurality of facial feature identifiers arranged in a pattern.

47. The method of claim 46, wherein the applying step comprises applying the beauty product to the facial feature of the user in a pattern corresponding to the pattern of the facial feature identifiers.

48. The method of claim 45, wherein the selecting step comprises matching the beauty product identifier with the facial feature identifier.

49. The method of claim 45, further comprising selecting an applicator comprising an applicator identifier matching the beauty product and facial feature identifiers.

50. A method of making a beauty template, the method comprising:
providing a facial feature on a sheet of material, the facial feature comprising at least one facial feature identifier matching an identifier of at least one beauty product.

51. The method of claim 50, wherein the facial feature comprises a plurality of facial feature identifiers arranged in a pattern corresponding to a pattern in which the at least one beauty product is to be applied to a corresponding facial feature of a user.

52. The method of claim 51, further comprising preselecting the at least one beauty product to provide the user with a targeted look.

53. A beauty product applicator comprising an applicator identifier, the beauty product applicator being configured for use with a beauty product having a beauty product identifier that matches the applicator identifier.

54. The beauty product applicator of claim 53, wherein the applicator identifier comprises one or more members selected from the group consisting of a letter, a number, a character, and a symbol.

55. The beauty product applicator of claim 53, wherein the beauty product applicator comprises a brush.

56. The beauty product applicator of claim 53, wherein the beauty product with which the beauty product applicator is configured to be used comprises one or more members selected from the group consisting of powder, foundation, blush, concealer, eyeshadow, lipstick, lipgloss, eyeliner, lipliner, mascara, and bronzer.
